(19) United States
(12) Patent Application Publication
(10) Pub. No.: US 2018/0214865 A1
(43) Pub. Date: Aug. 2, 2018

(54) POINT-OF-CARE DIAGNOSTIC CARTRIDGE HAVING A PROGRAMMABLE FLUIDIC WICKING NETWORK

(71) Applicant: Paratus Diagnostics, LLC, San Marcos, TX (US)

(72) Inventors: Zhenyuan LU, San Marcos, TX (US); John Jacob Carrano, San Marcos, TX (US); Roland Schneider, San Marcos, TX (US); John C. Carrano, San Marcos, TX (US)

(21) Appl. No.: 15/885,478

(22) Filed: Jan. 31, 2018

Related U.S. Application Data
(60) Provisional application No. 62/452,817, filed on Jan. 31, 2017.

(51) Int. Cl. B01L 3/00; C12Q 1/04

(52) U.S. Cl. CPC B01L 3/5023; C12Q 1/04; B01L 2400/0406; B01L 2200/06; B01L 2200/141; B01L 2200/16

(57) ABSTRACT

A specimen processing cartridge includes a first fluid source operable to deliver a first liquid to a first flow path and to transmit the first liquid to a testing area. The cartridge also includes a second fluid source operable to deliver a second liquid to a second flow path and a bridging member positioned between the first flow path and second flow path. The bridging member is operable to receive the second liquid from the second fluid source, and to deform from a first position to a second position upon receiving the second liquid. When not deformed, the bridging member does not contact the first flow path. When deformed, however, the bridging member contacts the first flow path and is operable to transmit the second liquid to the first flow path upon contact.

### TECHNICAL FIELD

The present disclosure relates generally to the field of medical diagnostics and more particularly to in vitro medical diagnostic devices including point-of-care in vitro medical diagnostic devices.

### BACKGROUND OF THE INVENTION

There is a recognized and compelling need for the rapid and accurate diagnosis of common infectious diseases in an out-patient setting. This need results from a rapidly emerging trend toward what is sometimes referred to as “patient centric care” in which convenience—along with better health outcomes and low cost—becomes a key market driver.

The field of in vitro diagnostics is well established, with many manufacturers and a wide spectrum of products and technologies. The testing for infectious pathogens in human patient specimens is, however, largely confined to centralized laboratory testing in Clinical Laboratory Improvement Amendment (CLIA) rated medium-complexity or high-complexity facilities. Commonplace techniques used in such laboratories include traditional culturing of specimens, immunological assaying using Enzyme-Linked Immunosorbent Assay (ELISA), nucleic acid testing (such as polymerase chain reaction, PCR), and other methods.

### BRIEF DESCRIPTION OF THE DRAWINGS

FIG. 1 is a perspective view of a specimen processing cartridge having a microfluidic substrate having a closable fluid flow path, wherein a cover of the specimen processing cartridge is removed to illustrate a fluid processing portion of the cartridge;

FIG. 2 is a top, detail view of a fluid processing portion of the specimen processing cartridge of FIG. 1;

FIGS. 3A-3C illustrate processes for operating the fluid processing portion of the specimen processing cartridge described with regard to FIG. 2;

FIGS. 4A and 4B, respectively, are a perspective view and side view of a portion of the microfluidic substrate and closeable fluid flow path;

FIG. 5 is a perspective view of a portion of a microfluidic flow path that includes a rectangular adhesive pad without a movable pad;

FIG. 6 is a perspective view of a portion of a microfluidic flow path that includes two parallel rectangular adhesive pads without a movable pad;

FIG. 7 is a perspective view of a portion of a microfluidic flow path that includes four linear-patterned, circular adhesive pad without a movable pad;

FIG. 8 is a perspective view of a portion of a microfluidic flow path that includes one rectangle adhesive pad without a movable pad;

FIG. 9 is a perspective view of a portion of a microfluidic flow path that includes three parallel rectangular adhesive pads without a movable pad;

FIG. 10 is a perspective view of a portion of a microfluidic substrate that includes two adhesive pads adhering a movable pad to the flow pad;

FIG. 11 is a perspective view of a portion of a microfluidic substrate that includes adhesive pads wrapped around a movable pad and a flow pad;

FIG. 12 is a perspective view of an alternative embodiment of a microfluidic flow path;

FIG. 13 is a side view of the microfluidic flow path of FIG. 12;

FIG. 14 is a perspective view of another alternative embodiment of a microfluidic flow path;

FIG. 15 is a side view of the microfluidic flow path of FIG. 14;

FIG. 16 is a front-perspective view of another alternative embodiment of a microfluidic flow path;

FIG. 17 is a rear-perspective view of the microfluidic flow path of FIG. 16;

FIG. 18 is a side view of the microfluidic flow path of FIG. 16; and

FIGS. 19-24 are perspective views of alternative embodiments of microfluidic flow paths.

### DETAILED DESCRIPTION

The conventional model for infectious disease diagnosis relies heavily on centralized laboratory testing (e.g. culture), which can often take two to four days to provide a reliable result. Applicant performed time-and-motion studies of medical practice and patient flow in the current model of infectious disease diagnosis and compared it to the new model relying on the devices described in this disclosure. A consequence of the conventional model is that patients are not necessarily properly diagnosed on their first visit or given the correct drug prescription. This results in money wasted on either incorrect or unnecessary prescriptions, inconvenience to patients owing to repeat visits, and even the potential for otherwise treatable illnesses to progress to more serious conditions requiring expensive hospital stays. In addition, it may contribute to the over-prescription of antibiotics, which is a cost burden to the healthcare system and may contribute to the increasing frequency of antibiotic resistant strains in the community.

There are some rapid diagnostic tests (RDTs) on the market today that are suitable for use in an out-patient setting. These RDTs, however, are simple “rule-in/rule-out” tests which do not necessarily inform clinical decision-making. Furthermore, many of these RDTs suffer from poor sensitivity and specificity, making the validity and clinical utility of their results dubious at best.

In diagnosing a patient, it is common for a physician to ask whether an illness is the consequence of a bacterial or a viral pathogen. The present disclosure relates to a system that is able to provide that answer during the patient visit with a high degree of accuracy. In this way, the correct diagnosis is obtained, and the best treatment option prescribed.

In point-of-care diagnostics for infectious disease, a premium is placed on the ability to achieve low complexity and low cost while substantially improving health outcomes. Further, to leverage the ubiquity of smartphones and other computing devices in common use globally, a specimen processing cartridge may facilitate the use of a computing device, such as a smart phone, to carry out a test for one or more pathogens.

The present disclosure relates to the manipulation of liquids within a specimen processing cartridge to implement processes associated with processing and analyzing a sample. More particularly, this disclosure relates to a microfluidic paper-based assay device that has a main flow channel with embedded reagents and several side channels for leading several specific reagents to wick from different reservoirs and flow into the main channel in a timed sequence. For lateral flow immunoassay, more than one different reagents may be introduced to the main channel based upon different methods. These reagents will flow into the main channel through a side channel at different times. For different reagents, it may be desirable to introduce the reagents at different times to have a sufficient reaction with embedded reagents on the main channel. Due to the connection between main channel and side channel, however, when reagents pass by a side channel, such reagents may also flow into the side channel by the capillary force. This “backflow” may result in waste of reagents and reduction of volume of fluid reacted with the embedded reagents, which may in turn affect the accuracy of lateral flow immunoassay. To mitigate this issue, an improved specimen processing device is disclosed that includes a structure that prevents backflow from the main channel and provides time delivery of fluids by utilizing wicking and gravitational forces.

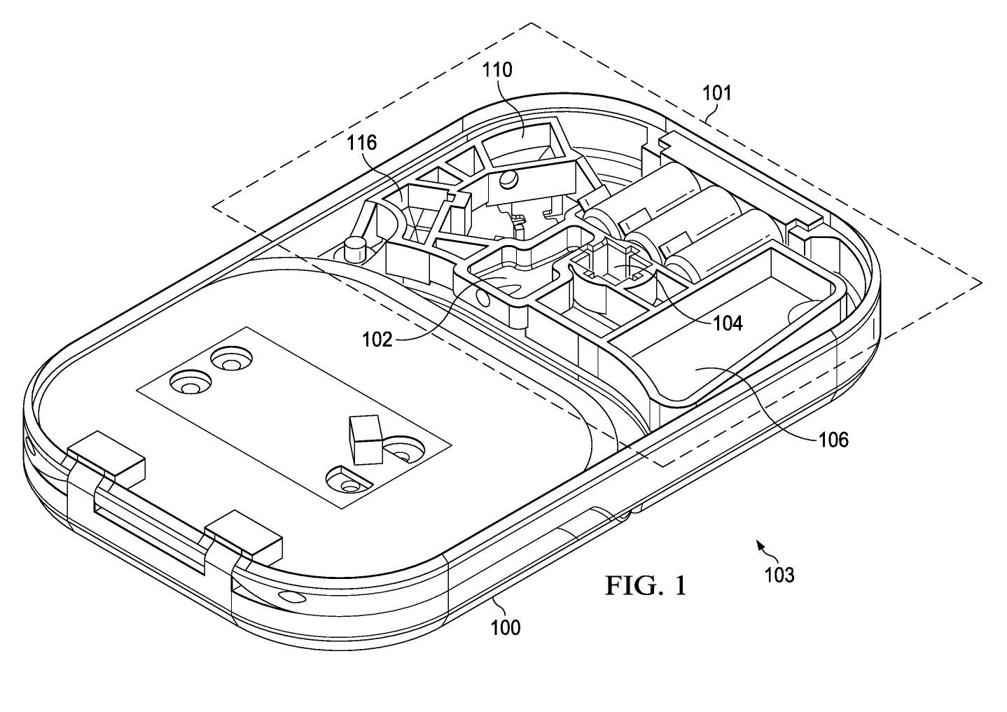

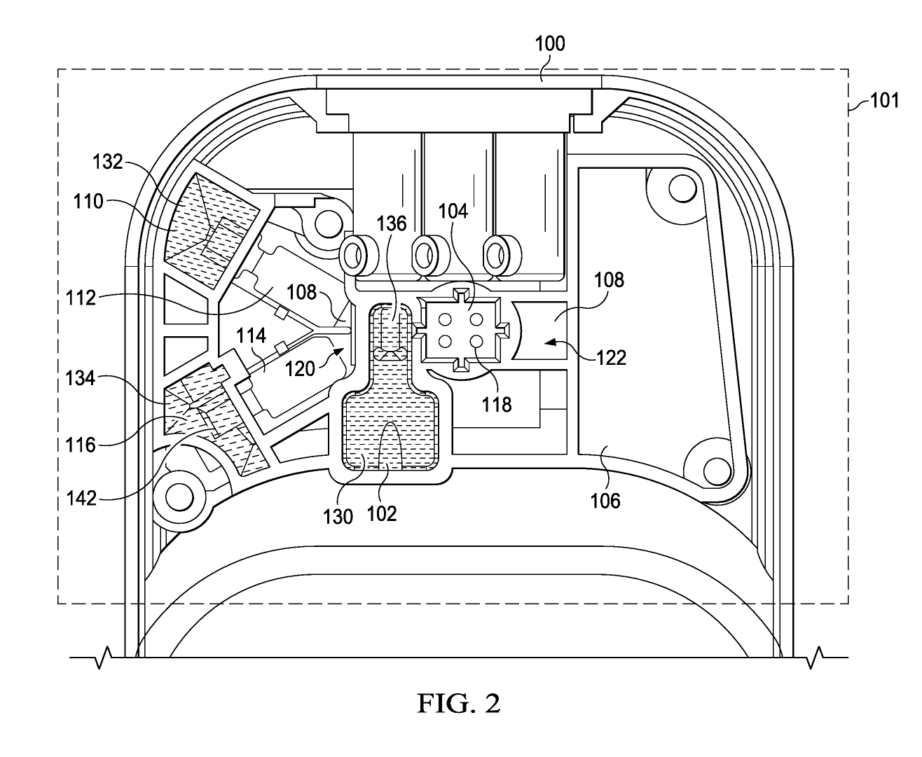

Referring now to the figures, FIGS. 1 and 2 show illustrative embodiments of a specimen processing cartridge 100 that includes a sample receiving portion 103 and a processing portion 101. The receiving portion 103 includes componentry for receiving a sample, which may be a tissue sample, a biological liquid sample, an environmental sample. The sample may be gathered using a sample collector, such as a swab, and subsequently extracted from the collector and suspended in a solution. The solution, including the sample, may be provided to the processing portion 101 of the specimen processing cartridge 100 at a first fluid source 102. To process the sample, the processing portion 101 may further include a microfluidic substrate 108 (that forms a part of a fluid flow circuit or flow path within the processing portion 101 of the specimen processing cartridge 100.

In some illustrative embodiments, the processing portion 101 encloses the substrate 108, which extends below the first fluid source 102 and underlies a bridging member at a first end 120 of a fluid flow path. In the illustrative embodiment, the substrate 108 underlies a first bridging member 112 and may also underlie a second bridging member 114 when the specimen processing cartridge is in an unactuated state. The substrate 108, first bridging member 112, and second bridging member 114 may be formed from a substrate material that demonstrates the ability to deform and conduct microfluidic flow. Examples of such materials include glass fiber, cellulose paper, nitrocellulose membrane, and combinations thereof. To that end, each of the substrate 108, first bridging member 112, and second bridging member 114, may have hydrophilic properties and may be selected so that, for example, the substrate 108 is more hydrophilic than the first bridging member 112, which may in turn be more hydrophilic than the second bridging member 114.

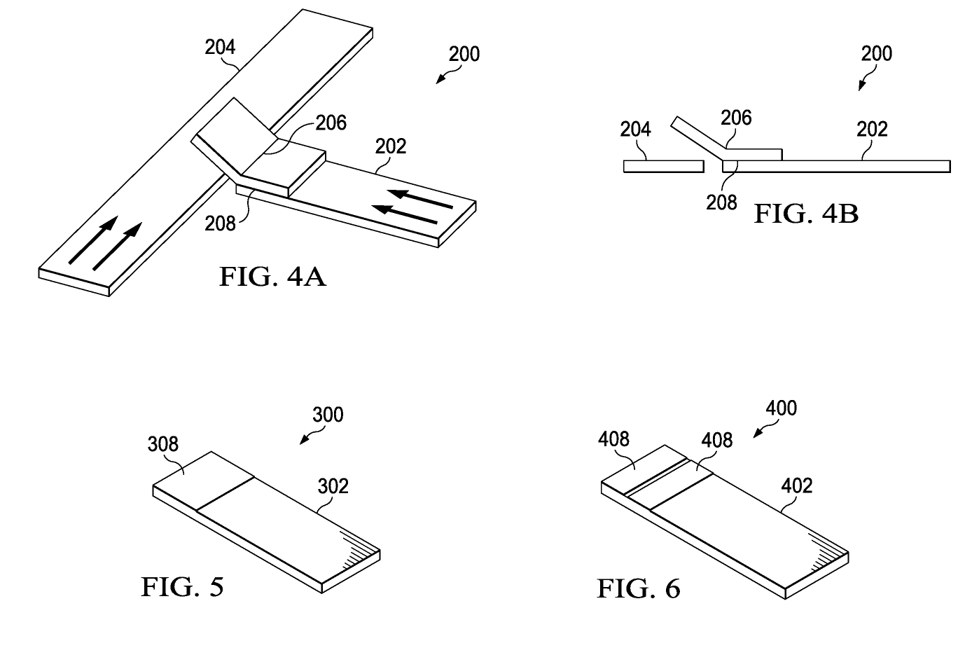

The flow path, defined by the substrate 108, extends from the first end 120 to a second end 122 of the fluid flow path that includes or is coupled to an absorbent reservoir 106. The absorbent reservoir 106 may be a sponge or similar material that conducts fluid from the substrate 108, thereby facilitating the drawing of fluid from the first end 120 toward the second end 122. Between the first end 120 and second end 122, the substrate 108 passes over a viewing area 104, which may be aligned with an inspection window or similar feature of the specimen processing cartridge 100 to allow viewing and analysis of the sample. Examples of bridging members that may function as the first bridging member 112 and second bridging member 114 are described in more detail with regard to FIGS. 4A-24.

Upon a first actuation event, a sample may be stripped from a collector and suspended in a first liquid 130 that is distributed to the substrate 108. The wicking properties of the substrate 108 may conduct the liquid toward the second end 122, thereby dispersing the first liquid 130 along with particles of the sample that are deposited across portions of the substrate 108, including the viewing area 104. In some embodiments, dried reagents 118 may be pre-deposited on (or affixed to) the substrate 108 at the viewing area 104 to attract and/or interact with particles of the sample as the first liquid 130 (which includes suspended sample) flows across the substrate 108.

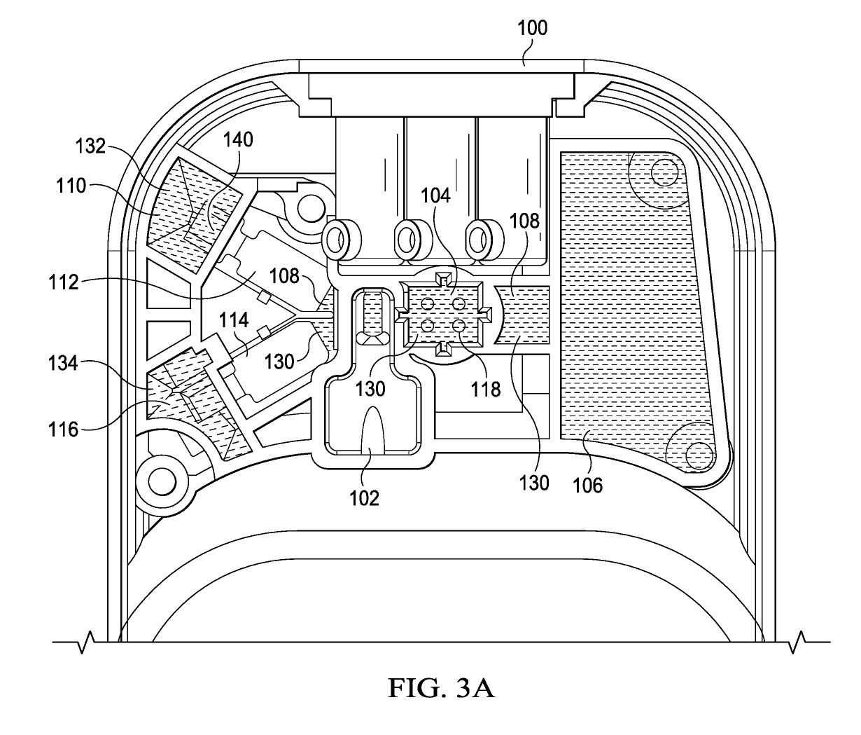

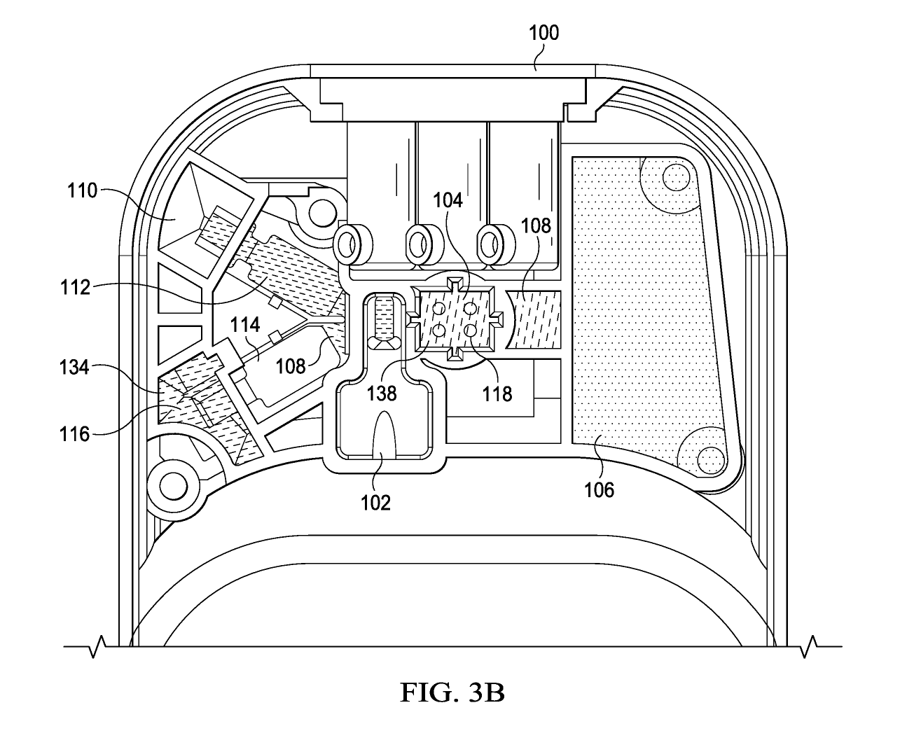

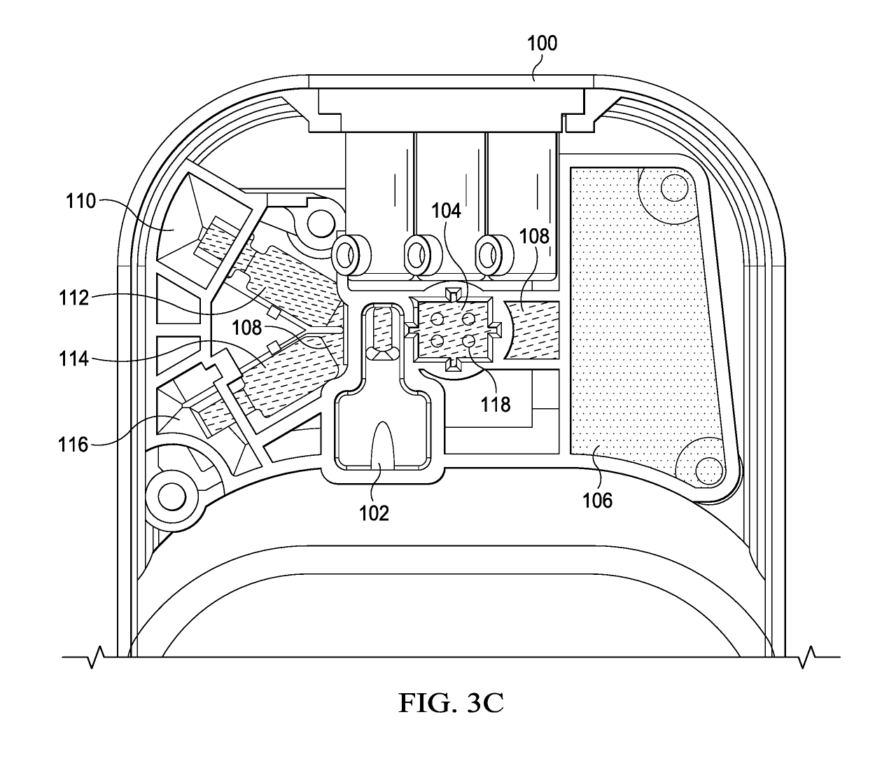

In subsequent processing steps, as described with regard to FIGS. 2-3C, the first bridging member 112 is operable to receive a second liquid 132 from a second fluid source 110. Prior to actuation, the second fluid source 110 may store a preselected volume of the second liquid 132. In addition, the first bridging member 112 is operable to deform and contact the first end 120 of the flow path, thereby allowing the second liquid 132 to be conducted across the substrate 108 toward the absorbent reservoir 106. The second liquid 132 may be a wash or an active solution that is operable to displace the first liquid and optionally to interact with sample particles or reagent at the viewing area 104. The second liquid 132 may include, for example, a lysing agent or a reagent that may interact with target pathogens to cause a reaction that reveals the presence of the target.

In embodiments that include the second bridging member 114 and a third fluid source 116, the second bridging member 114 may be operable to receive a third liquid 134 from the third fluid source 116. Prior to actuation, the third fluid source 116 may store a preselected volume of the third liquid. In addition, the second bridging member 114 is operable to deform and contact the first end 120 of the flow path, thereby allowing the third liquid 134 to be conducted across the substrate 108 toward the absorbent reservoir 106. The third liquid 134 may also be a wash or an active solution that is operable to displace the first liquid or second liquid 132 and optionally to interact with sample particles or reagent at the viewing area 104. Like the second liquid 132, the third liquid 134 may include, for example, a lysing agent or a reagent that may interact with target pathogens to cause a reaction that reveals the presence of the target. In some embodiments, the second liquid 132 or third liquid 134 (or a subsequent liquid) may be a liquid that facilitates viewing of the test carried out at the viewing area 104, such as a luminol peroxide.

Exemplary bridging members, analogous to bridging members 112 and 114, are described with regard to FIGS. 4A-24. In an illustrative embodiment, the bridging members include a flow pad that functions as a flow channel, an adhesive layer, and a foldable controlled pad. The foldable controlled pad may serve to prevent sample fluid from flowing back toward the flow pad and may also provide a time delay by virtue of its mechanical structure. The flow pad may be placed in contact with the foldable controlled pad using an adhesive pad that assists flow towards the foldable controlled pad. These attributes may be used to provide control over timing and to provide backflow prevention in the context of a specimen processing cartridge or a similar device. To that end, it is noted that the bridging members may be similarly operable to function in any device or system that includes timed supply of liquids or backflow prevention.

The substrates, bridging members, and components thereof may be formed using a microfluidic, paper-based material. An exemplary bridging member 200 is described with regard to FIGS. 4A and 4B. The bridging member 200 includes a movable pad 206 that is fabricated from glass fiber, cellulose paper, nitrocellulose membrane, or other suitable material. The movable pad 206 may be bent or otherwise deformed at an angle such that the movable pad 206 includes two flat portions angled relative toward one another and separated by a bend or a curve. The angle may range from, for example, 1° to 89°. When the bridging member 200 is used to provide a delayed supply of fluid, smaller angles may be associated with a shorter delay and larger angles may be associated with a longer delay as described in more detail below.

The movable pad 206 is coupled to a flow pad 202 by an adhesive pad 208 and, in the embodiment illustrated in FIGS. 4A and 4B, is separated from a substrate 204 or main channel by an air-gap. The air-gap between movable pad 206 and substrate 204 can prevent any backflows from the substrate 204 by eliminating contact between the movable pad 206 and substrate 204. Upon absorbing a liquid, the movable pad 206 swells and becomes heavier, resulting in gravitational forces overcoming internal strains within the movable pad 206 at the bend, thereby causing the movable pad 206 to flatten and touch the substrate 204. This process is not instantaneous, however, and a determinable amount of time elapsed between the time the movable pad 206 is exposed to the liquid and the time the movable pad 206 deforms to contact the substrate 204. The adhesive pad 208 provides stability by affixing a portion of the movable pad 206 to the flow pad 202. In some embodiments, the flow pad 202 and substrate 204 are formed from a nitrocellulose card having a specific flow or wicking rate.

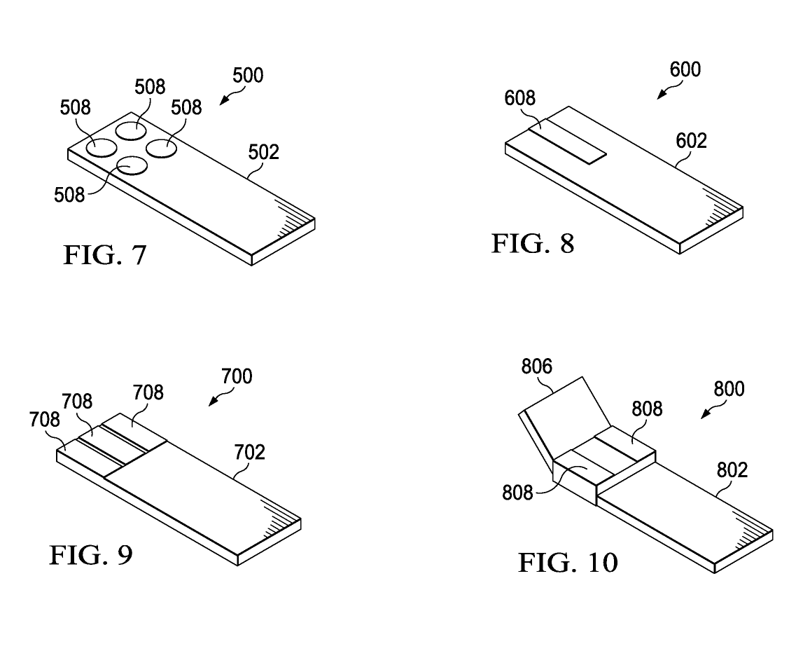

FIGS. 5-9 show alternative embodiments of a portion of the bridging member that include a flow pad and an adhesive portion that may be used to adhere a movable pad in the manner shown in FIGS. 4A and 4B. In the embodiment of FIG. 5, a base portion 300 of a bridging member includes a flow pad 302 and a rectangular adhesive pad 308. In the embodiment of FIG. 6, a base portion 400 of a bridging member includes a flow pad 402 and a rectangular adhesive pad 408. In the embodiment of FIG. 7, a base portion 500 of a bridging member includes a flow pad 502 and a plurality of circular adhesive pads 508. In the embodiment of FIG. 8, a base portion 600 of a bridging member includes a flow pad 602 and a rectangular adhesive pad strip 608. In the embodiment of FIG. 9, a base portion 700 of a bridging member includes a flow pad 702 and a plurality of rectangular adhesive pad strips 708. It is noted that where an adhesive is described with respect to any bridging portion referenced herein, the adhesive may be implemented in any of the configurations described above.

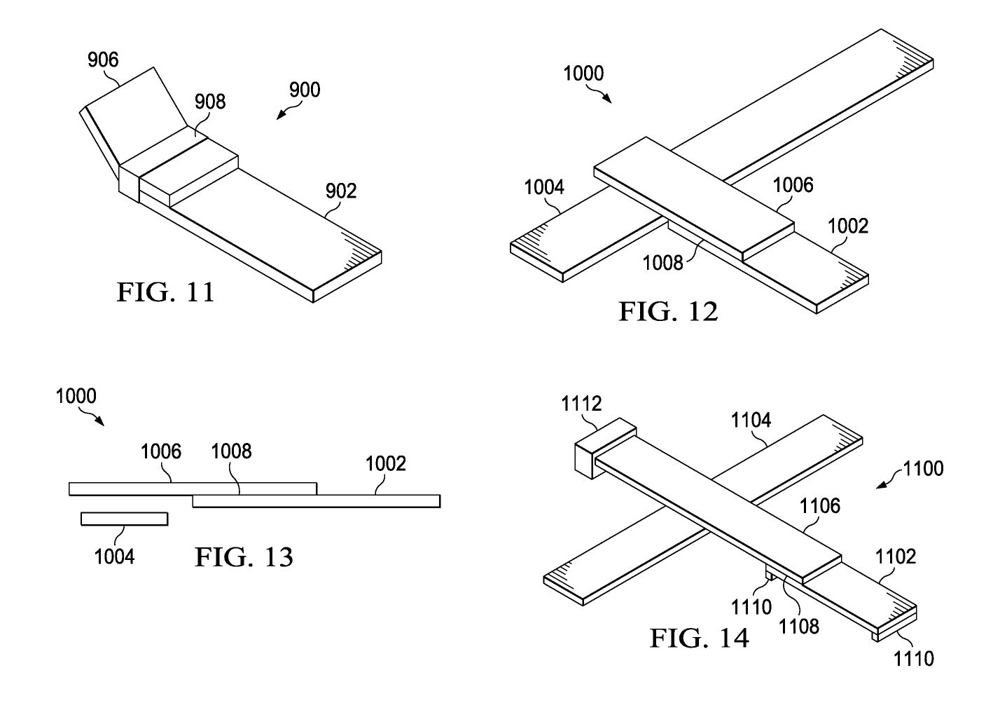

FIGS. 10 and 11 show alternative embodiments of a bridging member that include a base portion and a movable pad. In the embodiment of FIG. 10, the base portion 800 includes a flow pad 802 and a wrap-around adhesive strap 808 that partially wraps around a movable pad 806 to affix the movable pad 806 to the flow pad 802. In the embodiment of FIG. 11, the base portion 900 includes a flow pad 902 and a wrap-around adhesive strap 908 that encircles a movable pad 906 to affix the movable pad 906 to the flow pad 902.

FIGS. 12 and 13 show an alternative embodiment of a bridging member 1000 that includes a flat movable pad 1006 that is affixed to a flow pad 1002 by an adhesive 1008. The flow pad 1002 may be supported by flow pad supports (not shown, but analogous to supports 1110 shown in FIG. 14), which may be formed from any suitable nonabsorbent, or non-wicking material. The movable pad 1006 is supported at a first end by a contact area of the flow pad 1002, and is cantilevered over and offset from a substrate 1004 that acts as an intersecting flow channel. The bridging member is operable to receive a liquid at the flow pad 1002, which flows by wicking to the movable pad 1006. The movable pad 1006 is operable to receive the liquid and contact the substrate 1004 as a result of swelling, deflection (resulting from the weight of the liquid), or a combination thereof. Upon contacting the substrate 1004, the moving member is operable to transmit liquid from the flow pad 1002 to the substrate 1004.

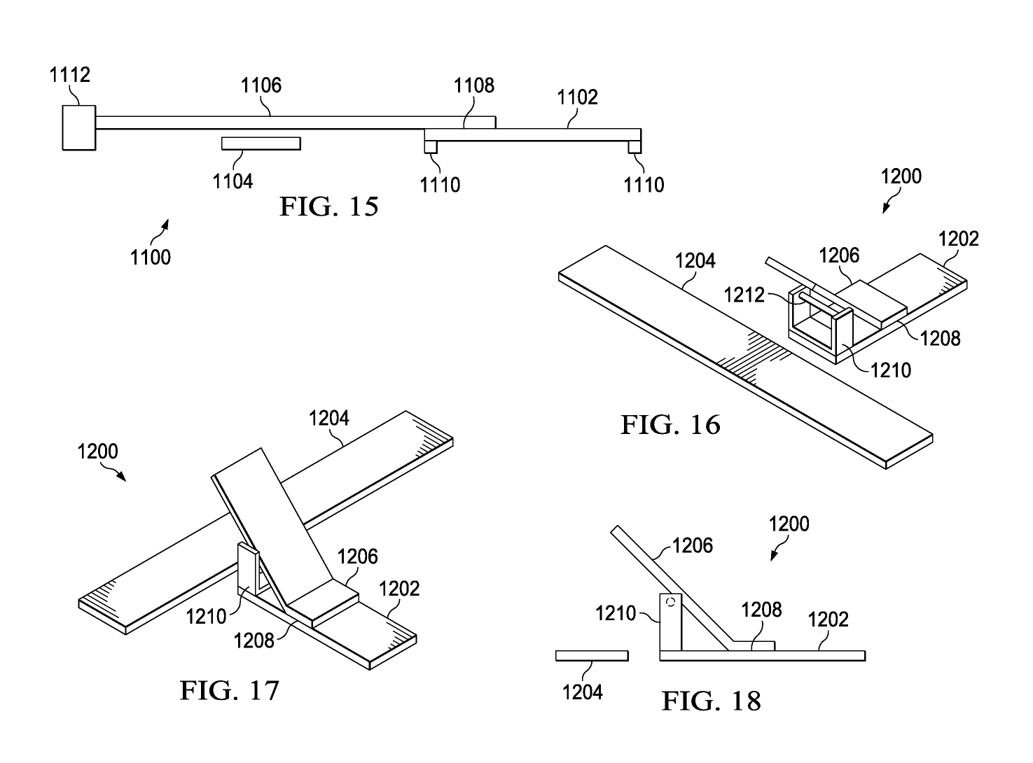

FIGS. 14 and 15 show an alternative embodiment of a bridging member 1100 that functions analogously to that of FIGS. 12 and 13, but does not include a cantilevered movable pad. The bridging member 1100 of FIG. 14 includes a flat movable pad 1106 that is affixed to a flow pad 1102 by an adhesive 1108. The flow pad 1102 is supported by flow pad supports 1110, which may be formed from any suitable nonabsorbent or non-wicking material. The movable pad 1106 is supported at a first end by a contact area of the flow pad 1102, and at a second, opposing end by an end support 1112, which may be materially or structurally similar to the flow pad supports 1110. A movable intermediate portion of the movable pad 1106 overlies and is offset from a substrate 1104 that acts as an intersecting flow channel. The bridging member is operable to receive a liquid at the flow pad 1102, which flows by wicking to the movable pad 1106. The movable pad 1106 is operable to receive the liquid and contact the substrate 1104 as a result of swelling, deflection (resulting from the weight of the liquid), or a combination thereof. Upon contacting the substrate 1104, the moving member is operable to transmit liquid from the flow pad 1102 to the substrate 1104.

Alternative embodiments of bridging members are described with regard to FIGS. 16-24. In the embodiment of FIGS. 16-18, a bridging member 1200 includes a movable pad 1206 that is supported at a first end by an interfacing portion with a flow pad 1202 (and joined by an adhesive 1208). The movable pad 1206 is initially supported in a raised position by pins 1212 ending under the movable pad 1206 and joined to a support 1210. The pins 1212 may be made from a glass fiber or other suitable materials and operate analogously to shear pins, and are operable to deform or fail in response to a movable pad 1206 weighted with liquid lowering onto the pins 1212. Failure of the pins 1212 allows the movable pad 1206 to lower into contact with the intersecting flow channel of the substrate 1204. In some embodiments the dimensions of the pins 1212 may be selected to increase, to varying degrees, the amount of time it takes for the movable pad 1206 to contact the substrate 1204 after receiving liquid from the flow pad 1202.

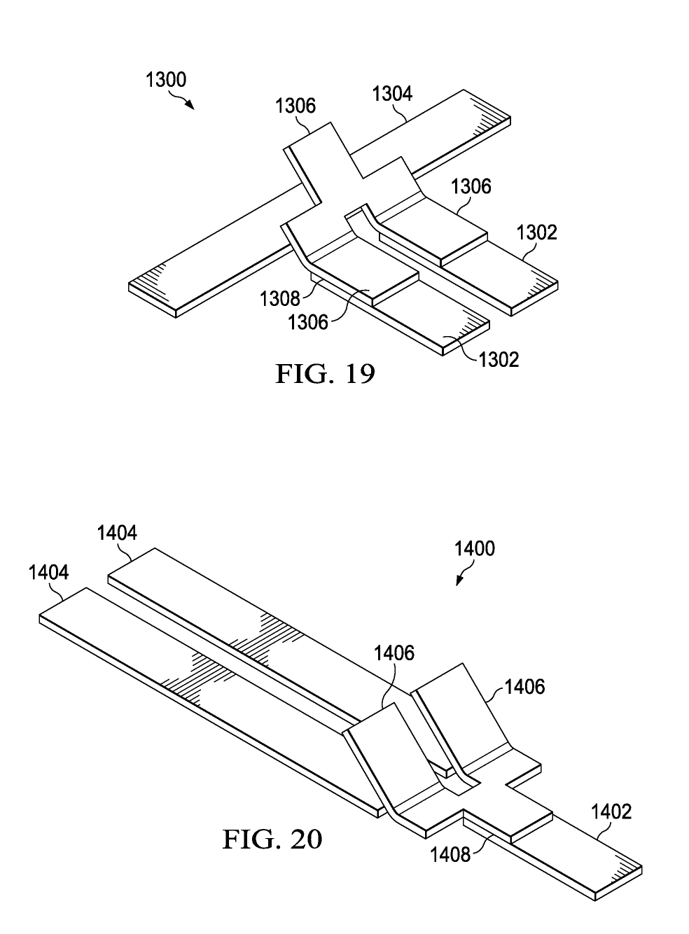

In the embodiment of FIG. 19, a bridging system 1300 includes a movable pad 1306 having parallel input channels that enable the input of liquid from two sources. To merge and/or mix liquid received from a fluid source, the movable pad 1306 has two inlet interfaces 1308, each of which is joined to a flow pad 1302 to receive a liquid. As liquids from each inlet are received at the movable pad 1306, the liquids mix and are wicked toward an outlet that contacts a substrate 1304 when liquids are received at, and weight down, the movable pad 1306. The embodiment of FIG. 20 is similar to that of FIG. 19 but reversed. The bridging member 1400 includes a flow pad 1402 joined to a movable pad 1406 at an inlet interface 1408 having two outlet paths that join to two separate flow channels (substrates 1404) when the movable pad 1406 is weighted by a liquid. Rather than provide for mixing, the embodiment generates two similar fluid flow paths that may facilitate the application of disparate testing or analysis processes to each of the flow channels.

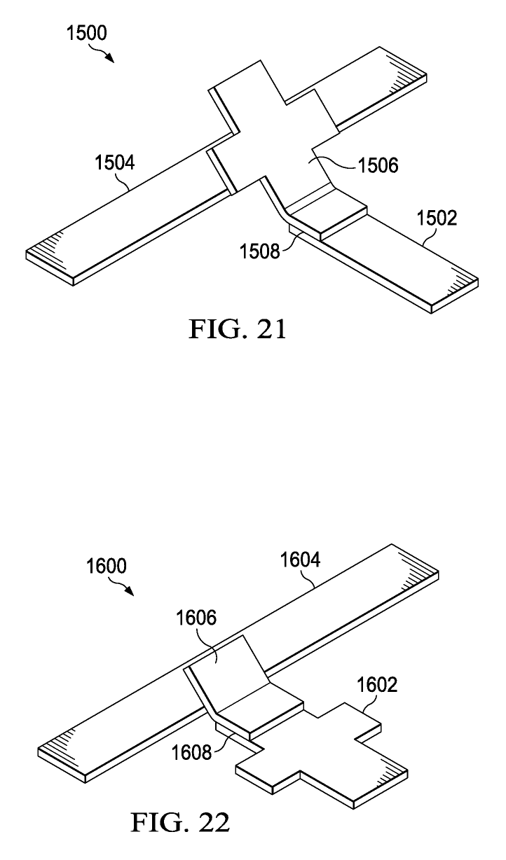

In the embodiment of FIG. 21, a bridging system 1500 includes a movable pad 1506 that has a wider intermediate area. The wider intermediate area may take additional time to wick, thereby slowing the rate at which liquid wicks from the flow pad 1502 and inlet interface 1508 across the movable pad 1506 to the substrate 1504. The bridging system 1600 of FIG. 22 is analogous to that of FIG. 21 with the exception that the wider intermediate area is removed from the movable pad 1606 and is instead included at the input flow pad 1602 to delay flow across the inlet interface 1608 to the movable pad 1606 and subsequently to the substrate 1604.

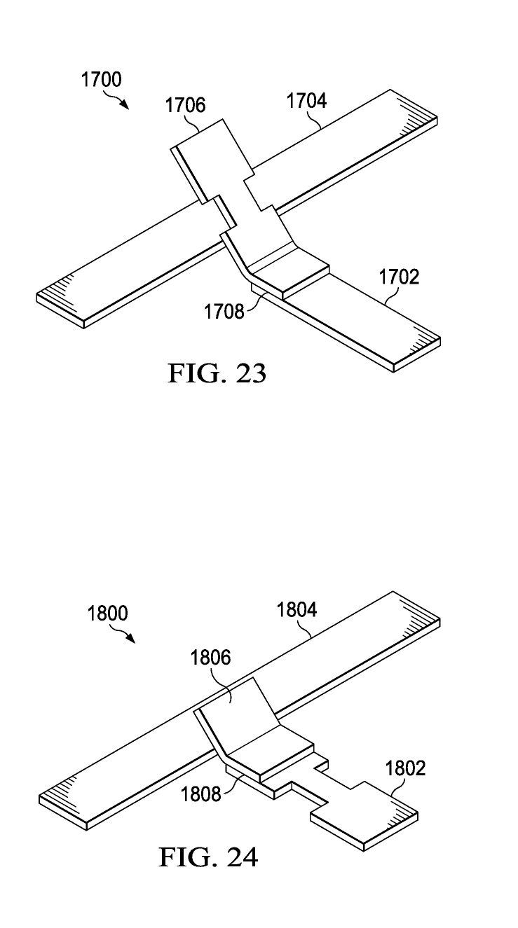

The bridging system 1700 of the embodiment of FIG. 23 is similar to that of FIG. 21, but differs in that the wider intermediate area of movable pad 1706 has been replaced by a narrowed intermediate area. In contrast to a wider area, the narrowed area will saturate more quickly and therefore the movable pad 1706 of FIG. 23 will act more quickly to transmit liquid from the flow pad 1702 and inlet interface 1708 to the flow channel of the substrate 1704. The embodiment of FIG. 24 is analogous to that of FIG. 23 with the exception that the narrowed intermediate area is removed from the moving member 1806 and is instead included at the input flow pad 1802.

Referring again to FIGS. 1, 2, and 3A-3C, in accordance with the foregoing embodiments, a specimen processing cartridge 100 is disclosed that includes a first fluid source 102 operable to deliver a first liquid 130 to a first flow path. The first fluid source 102 may be positioned downstream of a sample collector, and may therefore receive and retain the first liquid after it has been circulated over a sample or specimen such that the first liquid includes sample particles when it reaches the first fluid source 102. The first flow path is operable to transmit the first liquid 130 to a testing or viewing area 104 by wicking, absorption, or a combination thereof. The specimen processing cartridge 100 further includes a second fluid source 110 that is operable to deliver a second liquid 132 to a second flow path. In addition, the specimen processing cartridge 100 includes a bridging member 112, which may be a first bridging member, positioned within the second flow path proximate to the first flow path. The bridging member 112 is operable to receive the second liquid 132 from the second fluid source 110, and is further operable to deform from a first position to a second position upon receiving the second liquid 132. The bridging member 112 does not contact a substrate 108 that forms a portion of the first flow path when in the first position, but does contact the substrate 108 (and flow path) when in the second position. The bridging member 112 may be operable to transition from the first position to the second position by virtue of increased weight or swelling associated with a liquid being applied to a movable pad of the bridging member 112. After moving to the second position, the bridging member 112 is operable to transmit the second liquid 132 to the substrate 108 of the first flow path upon contact.

The specimen processing cartridge 100 may include any reasonable number of additional fluid sources and bridging members. For example, the specimen processing cartridge 100 may further include a third fluid source 116 source operable to deliver a third liquid 134 to a third flow path that includes a second bridging member 114. The second bridging member 114 may be positioned along the third flow path and proximate to the first flow path, and may be operable to receive the third liquid 134 from the third fluid source 116. Like the bridging member 112, the second bridging member 114 is operable to deform from a first position to a second position upon receiving the third liquid 134. The second bridging member 114 does not contact the substrate 108 of the first flow path when in the first position but does contact the substrate 108 of the first flow path when in the second position. Further, the second bridging member 114 is operable to transmit the third liquid 134 to the substrate 108 of the first flow path upon contact.

In some embodiments, the substrate 108 is a microfluidic substrate selected from the group consisting of a glass fiber, cellulose paper, nitrocellulose membrane, and a combination thereof. The substrate 108 is thereby operable to transport liquid by absorption, wicking, or a combination thereof. In some embodiments, the first end 120 is proximate to the bridging member 112, and a second end 122 of the flow path is coupled to an absorbent reservoir 106, and an intermediate portion that is proximate to a test area or viewing area 104. In some embodiments, pre-dried reagent 118 is placed at the intermediate portion and operable to react with one of the first fluid, second fluid, third fluid, and/or sample upon exposure.

In some embodiments, the second flow path comprises a flow pad coupled to a fluid outlet of the second fluid source 110. The flow pad may be affixed to or formed integrally with a movable pad of the bridging member 112. Alternatively, the bridging member 112 may comprise a movable pad that is fixed to the flow pad by an adhesive. The movable pad of the bridging member 112 may be bent at a preselected angle when the bridging member is in the first position, and the movable pad may be operable to deform to a flattened or more flattened state to contact the first end 120 of the substrate 108 of the first flow path upon receiving the second liquid 132.

In embodiments in which the movable pad of the bridging member 112 is affixed to a flow pad by an adhesive, the adhesive may be in the form of rectangular adhesive pads positioned between the flow pad and the movable pad, a plurality of circular adhesive pads positioned between the flow pad and the movable pad, one or more adhesive pads wrapped around the flow pad and the movable pad, or any combination thereof.

In some embodiments, the bridging member 112 is offset from the first end 120 of the substrate 108 of the first flow path when the bridging member 112 is in the first position. In such embodiments, the bridging member 112 is operable to swell or wick a liquid received from the second fluid source. Such swelling causes the bridging member 112 to either expand from the first position to the second position or to become weighted down to move to the second position in which the bridging member 112 contacts the substrate 108. The bridging member 112 may be deployed in any of the embodiments described with regard to FIGS. 4A-24, and may also be formed integrally with a flow pad.

The specimen processing cartridge 100 may be used to implement any number of suitable methods for processing a liquid that includes a sample. In some embodiments, an illustrative method for detecting a target using the specimen processing cartridge 100 includes delivering the first liquid 130 from the first fluid source 102 to a wicking substrate 108 defining a first flow path. The first liquid may be delivered by manually or automatically actuating a valve, rupturing a seal, or otherwise opening a fluid pathway, which may be a first outlet 136 that separates the first fluid source 102 from the substrate 108. The first fluid may wick across the substrate 108 and toward the absorbent reservoir, which may augment the wicking potential of the substrate 108 by absorbing fluid therefrom.

In an embodiment in which the specimen processing cartridge 100 includes the third fluid source 116, the method may further include delivering the third liquid 134 from the third fluid source 116 to the third flow path by actuating a third outlet 142. The third liquid 134 may also be delivered by manually or automatically actuating a valve, rupturing a seal, or otherwise opening a fluid pathway, which may be the third outlet 142, which separates the third fluid source 116 from a third flow path that includes at least the second bridging member 114. Delivery of the third liquid 134 to the second bridging member 114 causes the second bridging member 114 to deform and contact the first flow path at the first end 120 of the substrate 108. As shown in FIG. 3C, contact between the second bridge member 114 and the substrate 108 results in flow of the third liquid 134 to the first flow path, where the third fluid 134 may interact with the substrate fluid 138 in a manner similar to the second fluid 132 (by mixing or displacement). In such embodiments, the method may further include analyzing the viewing area 104 where the first liquid 130 (including the sample), second liquid 132, and/or third liquid 134 may have interacted with one another or with one or more pre-dried reagents 118 to determine whether a target substance or pathogen was present in the sample. The foregoing method may be iterative and to that end, may involve any number of secondary liquids using bridging members in the manner described to achieve an ordered and timed sequence of liquid interactions. For example, in some embodiments, the second liquid 132 may be a wash fluid and the third liquid 134 may include a reagent.

In some embodiments, each of the first outlet 136, second outlet 140, and (if present) the third outlet 142 may be actuated simultaneously, and the sequenced interaction of the first liquid 130, second liquid 132, and third liquid 134 may be determined by the configuration and composition of the bridging member 112 and second bridging member 114, as described above. In such embodiments, the steps of delivering the first liquid 130 to the first flow path, delivering the second liquid 132 to the second flow path, and delivering the third liquid 134 to the third flow path may occur simultaneously. In such embodiments, the first bridging member 112 may contact the substrate 108 of the first flow path after a first time delay following the delivery of the second liquid 132 to the second flow path, and the second bridging member 114 may contact the substrate 108 of the first flow path after a second time delay following the delivery of the third liquid 134 to the third flow path. In such embodiments, the second time delay may be less than, greater than or equal to the first time delay depending on the desired sequence of interaction.

It is noted that unless an embodiment is expressly stated as being incompatible with other embodiments, the concepts and features described with respect to each embodiment may be applicable to and applied in connection with concepts and features described in the other embodiments without departing from the scope of this disclosure. To that end, the above-disclosed embodiments have been presented for purposes of illustration and to enable one of ordinary skill in the art to practice the disclosure, but the disclosure is not intended to be exhaustive or limited to the forms disclosed. Many insubstantial modifications and variations will be apparent to those of ordinary skill in the art without departing from the scope and spirit of the disclosure. The scope of the claims is intended to broadly cover the disclosed embodiments and any such modification, including without limitation the following examples.

### EXAMPLES

### Example 1

A specimen processing cartridge comprising:
a first fluid source operable to deliver a first liquid to a first flow path and to transmit the first liquid to a testing area;
a second fluid source operable to deliver a second liquid to a second flow path; and
a bridging member positioned between the first flow path and second flow path, wherein the bridging member is operable to receive the second liquid from the second fluid source, and wherein the bridging member is operable to deform from a first position to a second position upon receiving the second liquid,
wherein the bridging member does not contact the first flow path when in the first position and wherein the bridging member contacts the first flow path when in the second position, and
wherein the bridging member is operable to transmit the second liquid to the first flow path upon contacting the first flow path.

### Example 2

The specimen processing cartridge of example 1, wherein the bridging member is a first bridging member, the specimen processing cartridge further comprising:
a third fluid source operable to deliver a third liquid to a third flow path; and
a second bridging member positioned between the first flow path and third flow path, wherein the second bridging member is operable to receive the third liquid from the third fluid source, and wherein the second bridging member is operable to deform from a first position to a second position upon receiving the third liquid,
wherein the second bridging member does not contact the first flow path when in the first position and wherein the second bridging member contacts the first flow path when in the second position, and
wherein the second bridging member is operable to transmit the third liquid to the first flow path upon contacting the first flow path.

### Example 3

The specimen processing cartridge of example 1, wherein the first fluid flow path comprises a microfluidic substrate selected from the group consisting of a glass fiber, cellulose paper, nitrocellulose membrane, and a combination thereof, and wherein the microfluidic substrate is operable to transport liquid by wicking.

### Example 4

The specimen processing cartridge of example 1, wherein the first fluid flow path comprises a first end proximate to the bridging member, a second end that is coupled to an absorbent reservoir, and an intermediate portion that is proximate to a test area.

### Example 5

The specimen processing cartridge of example 4, wherein the intermediate portion comprises a pre-dried reagent.

### Example 6

The specimen processing cartridge of example 1, wherein the second flow path comprises a flow pad coupled to a fluid outlet of the second fluid source, and wherein the bridging member comprises a movable pad fixed to the flow pad by an adhesive.

### Example 7

The specimen processing cartridge of example 6, wherein the movable pad is bent at a preselected angle when the bridging member is in the first position, and wherein the movable pad is operable to deform to cause movement of the bridging member from the first position to the second position upon receiving the second liquid from the flow pad.

### Example 8

The specimen processing cartridge of example 6, wherein the adhesive is selected from the group consisting of one or more rectangular adhesive pads positioned between the flow pad and the movable pad, a plurality of circular adhesive pads positioned between the flow pad and the movable pad, and one or more adhesive pads wrapped around the flow pad and the movable pad.

### Example 9

The specimen processing cartridge of example 1, wherein the first fluid flow path is coupled to an outlet of the first fluid source, and wherein the first liquid comprises a sample.

### Example 10

The specimen processing cartridge of example 1, wherein a portion of the bridging member is offset from the first flow path when the bridging member is in the first position, and wherein the bridging member is operable to swell upon absorbing fluid from the second fluid source, such swelling causing the bridging member to expand from the first position to the second position.

### Example 11

The specimen processing cartridge of example 1, wherein a portion of the bridging member is offset from the first flow path when the bridging member is in the first position, and wherein the bridging member is operable to receive the second liquid from the second fluid source, and wherein the bridging member is configured to move from the first position to the second position when subjected to the weight of the second liquid.

### Example 12

The specimen processing cartridge of example 1, wherein the bridging member comprises a movable pad having a first portion that is supported by and coupled to a flow pad, and a second portion overlies and is offset from the first flow path when the bridging member is in the first position.

### Example 13

The specimen processing cartridge of example 1, wherein the bridging member comprises a movable pad having a wider intermediate area between a first end and a second end.

### Example 14

The specimen processing cartridge of example 1, wherein the bridging member comprises a movable pad having a narrowed intermediate area between a first end and a second end.

### Example 15

A method for detecting a target using a specimen processing cartridge comprising a first fluid source, a second fluid source, a first flow path, and a second flow path, and a bridging coupled to the second flow path and offset from the first flow path when the bridging member is in a first position, the method comprising:
delivering a first liquid from the first fluid source to the first flow path
delivering a second liquid from the second fluid source to the second flow path, wherein delivering the second liquid comprises flowing the second liquid from the second flow path to the bridging member, thereby causing the bridging member to deform and contact the first flow path, and wherein the bridge member contacting the first flow path results in flow of the second liquid to the first flow path.

### Example 16

The method of example 15, wherein coupling the second fluid flow path to the first fluid flow path comprises mixing the second liquid with the first liquid.

### Example 17

The method of example 15, wherein the bridging member is a first bridging member, and wherein the specimen processing cartridge further comprises a third flow path and a second bridging member coupled to the third flow path and offset from the first flow path when the bridging member is in the first position, the method further comprising:
delivering a third liquid from the third fluid source to the third flow path, wherein delivering the third liquid comprises flowing the third liquid from the third flow path to the second bridging member, thereby causing the second bridging member to deform and contact the first flow path, and wherein the second bridge member contacting the first flow path results in flow of the third liquid to the first flow path.

### Example 18

The method of example 17, wherein the first liquid comprises a sample, the second liquid comprises a wash, and the third liquid comprises a reagent.

### Example 19

The method of example 17, wherein the steps of delivering the first liquid to the first flow path, delivering the second liquid to the second flow path, and delivering the third liquid to the third flow path occur simultaneously.

### Example 20

The method of example 19, wherein the first bridging member contacts the first flow path after a first time delay following the delivery of the second fluid to the second flow path, and wherein the second bridging member contacts the first flow path after a second time delay following the delivery of the third fluid to the third flow path, and wherein the second time delay is greater than the first time delay.

### Example 21

A specimen processing cartridge comprising:
a first fluid source operable to deliver a first liquid to a first flow path; and
a bridging member positioned between the first flow source and an isolated portion of the first flow path, wherein the bridging member is operable to receive the first liquid from the first fluid source, and wherein the bridging member is operable to deform from a first position to a second position upon receiving the first liquid,
wherein the bridging member does not contact the isolated portion of the first flow path when in the first position and wherein the bridging member contacts the isolated portion of the first flow path when in the second position, and
wherein the bridging member is operable to transmit the first liquid to the isolated portion of the first flow path upon contacting the isolated portion of the first flow path.

### Example 22

The specimen processing cartridge of example 21, wherein the bridging member is a first bridging member, the specimen processing cartridge further comprising:
a second fluid source operable to deliver a second liquid to a second flow path; and
a second bridging member positioned between the isolated portion of the first flow path and the second flow path, wherein the second bridging member is operable to receive the second liquid from the second fluid source, and wherein the second bridging member is operable to deform from a first position to a second position upon receiving the second liquid,
wherein the second bridging member does not contact the isolated portion of the first flow path when in the first position and wherein the second bridging member contacts the isolated portion of the first flow path when in the second position, and
wherein the second bridging member is operable to transmit the second liquid to the isolated portion of the first flow path upon contacting the isolated portion of the first flow path.

### Example 23

The specimen processing cartridge of example 21, wherein the first fluid flow path comprises a microfluidic substrate selected from the group consisting of a glass fiber, cellulose paper, nitrocellulose membrane, and a combination thereof, and wherein the microfluidic substrate is operable to transport liquid by wicking.

### Example 24

The specimen processing cartridge of example 21, wherein the isolated portion of the first fluid flow path is coupled to an absorbent reservoir, and wherein the isolated portion of the first fluid flow path comprises an intermediate portion that is proximate to a test area.

### Example 25

The specimen processing cartridge of example 24, wherein the intermediate portion comprises a pre-dried reagent.

### Example 26

The specimen processing cartridge of example 21, wherein the first flow path comprises a flow pad coupled to a fluid outlet of the first fluid source, and wherein the bridging member comprises a movable pad fixed to the flow pad by an adhesive.

### Example 27

The specimen processing cartridge of example 26, wherein the movable pad is bent at a preselected angle when the bridging member is in the first position, and wherein the movable pad is operable to deform to cause movement of the bridging member from the first position to the second position upon receiving the second liquid from the flow pad.

### Example 28

The specimen processing cartridge of example 26, wherein the adhesive is selected from the group consisting of one or more rectangular adhesive pads positioned between the flow pad and the movable pad, a plurality of circular adhesive pads positioned between the flow pad and the movable pad, and one or more adhesive pads wrapped around the flow pad and the movable pad.

### Example 29

The specimen processing cartridge of example 21, wherein a portion of the bridging member is offset from the first flow path when the bridging member is in the first position, and wherein the bridging member is operable to swell upon absorbing fluid from the first fluid source, such swelling causing the bridging member to expand from the first position to the second position.

As used herein, the singular forms “a”, “an” and “the” are intended to include the plural forms as well, unless the context clearly indicates otherwise. It will be further understood that the terms “comprise” and/or “comprising,” when used in this specification and/or the claims, specify the presence of stated features, steps, operations, elements, and/or components, but do not preclude the presence or addition of one or more other features, steps, operations, elements, components, and/or groups thereof. In addition, the steps and components described in the above embodiments and figures are merely illustrative and do not imply that any particular step or component is a requirement of a claimed embodiment.

1. A specimen processing cartridge comprising:
a first fluid source operable to deliver a first liquid to a first flow path and to transmit the first liquid to a testing area;
a second fluid source operable to deliver a second liquid to a second flow path; and
a bridging member positioned between the first flow path and second flow path, wherein the bridging member is operable to receive the second liquid from the second fluid source, and wherein the bridging member is operable to deform from a first position to a second position upon receiving the second liquid,
wherein the bridging member does not contact the first flow path when in the first position and wherein the bridging member contacts the first flow path when in the second position, and
wherein the bridging member is operable to transmit the second liquid to the first flow path upon contacting the first flow path.

2. The specimen processing cartridge of claim 1, wherein the bridging member is a first bridging member, the specimen processing cartridge further comprising:
a third fluid source operable to deliver a third liquid to a third flow path; and
a second bridging member positioned between the first flow path and third flow path, wherein the second bridging member is operable to receive the third liquid from the third fluid source, and wherein the second bridging member is operable to deform from a first position to a second position upon receiving the third liquid,
wherein the second bridging member does not contact the first flow path when in the first position and wherein the second bridging member contacts the first flow path when in the second position, and
wherein the second bridging member is operable to transmit the third liquid to the first flow path upon contacting the first flow path.

3. The specimen processing cartridge of claim 1, wherein the first fluid flow path comprises a microfluidic substrate selected from the group consisting of a glass fiber, cellulose paper, nitrocellulose membrane, and a combination thereof, and wherein the microfluidic substrate is operable to transport liquid by wicking.

4. The specimen processing cartridge of claim 1, wherein the first fluid flow path comprises a first end proximate to the bridging member, a second end that is coupled to an absorbent reservoir, and an intermediate portion that is proximate to a test area.

5. The specimen processing cartridge of claim 4, wherein the intermediate portion comprises a pre-dried reagent.

6. The specimen processing cartridge of claim 1, wherein the second flow path comprises a flow pad coupled to a fluid outlet of the second fluid source, and wherein the bridging member comprises a movable pad fixed to the flow pad by an adhesive.

7. The specimen processing cartridge of claim 6, wherein the movable pad is bent at a preselected angle when the bridging member is in the first position, and wherein the movable pad is operable to deform to cause movement of the bridging member from the first position to the second position upon receiving the second liquid from the flow pad.

8. The specimen processing cartridge of claim 6, wherein the adhesive is selected from the group consisting of one or more rectangular adhesive pads positioned between the flow pad and the movable pad, a plurality of circular adhesive pads positioned between the flow pad and the movable pad, and one or more adhesive pads wrapped around the flow pad and the movable pad.

9. The specimen processing cartridge of claim 1, wherein the first fluid flow path is coupled to an outlet of the first fluid source, and wherein the first liquid comprises a sample.

10. The specimen processing cartridge of claim 1, wherein a portion of the bridging member is offset from the first flow path when the bridging member is in the first position, and wherein the bridging member is operable to swell upon absorbing fluid from the second fluid source, such swelling causing the bridging member to expand from the first position to the second position.

11. The specimen processing cartridge of claim 1, wherein a portion of the bridging member is offset from the first flow path when the bridging member is in the first position, and wherein the bridging member is operable to receive the second liquid from the second fluid source, and wherein the bridging member is configured to move from the first position to the second position when subjected to the weight of the second liquid.

12. The specimen processing cartridge of claim 1, wherein the bridging member comprises a movable pad having a first portion that is supported by and coupled to a flow pad, and a second portion overlies and is offset from the first flow path when the bridging member is in the first position.

13. The specimen processing cartridge of claim 1, wherein the bridging member comprises a movable pad having a wider intermediate area between a first end and a second end.

14. The specimen processing cartridge of claim 1, wherein the bridging member comprises a movable pad having a narrowed intermediate area between a first end and a second end.

15. A method for detecting a target using a specimen processing cartridge comprising a first fluid source, a second fluid source, a first flow path, and a second flow path, and a bridging coupled to the second flow path and offset from the first flow path when the bridging member is in a first position, the method comprising:
delivering a first liquid from the first fluid source to the first flow path
delivering a second liquid from the second fluid source to the second flow path, wherein delivering the second liquid comprises flowing the second liquid from the second flow path to the bridging member, thereby causing the bridging member to deform and contact the first flow path, and wherein the bridge member contacting the first flow path results in flow of the second liquid to the first flow path.

16. The method of claim 15, wherein coupling the second fluid flow path to the first fluid flow path comprises mixing the second liquid with the first liquid.

17. The method of claim 15, wherein the bridging member is a first bridging member, and wherein the specimen processing cartridge further comprises a third flow path and a second bridging member coupled to the third flow path and offset from the first flow path when the bridging member is in the first position, the method further comprising:
delivering a third liquid from the third fluid source to the third flow path, wherein delivering the third liquid comprises flowing the third liquid from the third flow path to the second bridging member, thereby causing the second bridging member to deform and contact the first flow path, and wherein the second bridge member contacting the first flow path results in flow of the third liquid to the first flow path.

18. The method of claim 17, wherein the first liquid comprises a sample, the second liquid comprises a wash, and the third liquid comprises a reagent.

19. The method of claim 17, wherein the steps of delivering the first liquid to the first flow path, delivering the second liquid to the second flow path, and delivering the third liquid to the third flow path occur simultaneously.

20. The method of claim 19, wherein the first bridging member contacts the first flow path after a first time delay following the delivery of the second fluid to the second flow path, and wherein the second bridging member contacts the first flow path after a second time delay following the delivery of the third fluid to the third flow path, and wherein the second time delay is greater than the first time delay.
